(19) United States
(12) Patent Application Publication
(10) Pub. No.: US 2008/0114079 A1
(43) Pub. Date: May 15, 2008

(54) TRPM2-specfic inhibitors

(76) Inventors: Frances E. Lund, Saranac Lake, NY (US); Santiago Partida-Sanchez, Galloway, OH (US); Tim Walseth, Roseville, MN (US)

Correspondence Address: KENYON & KENYON LLP, ONE BROADWAY, NEW YORK, NY, 10004, US

(21) Appl. No.: 11/652,265

(22) Filed: Jan. 10, 2007

Related U.S. Application Data
(60) Provisional application No. 60/757,980, filed on Jan. 10, 2006.

(51) Int. Cl. A61K 47/00; C12N 5/06; C12Q 1/02; A61P 9/00; A61P 1/00; A61P 19/00

(52) U.S. Cl. 514/789; 435/375; 435/29

(57) ABSTRACT

The present invention relates to methods and compositions for modulating ADPR-mediated migratory activity of cells through regulation of the TRPM2 cation channel. Such methods and compositions may be used for the treatment of disorders including, but not limited to, inflammation, ischemia, atherosclerosis, asthma, autoimmune disease, diabetes, arthritis, allergies, and transplant rejection. Such cells include, for example, neutrophils, lymphocytes, eosinophils, macrophages, monocytes and dendritic cells. The invention further relates to specific inhibition of TRPM2 by blocking the activity of ADPR. The invention also relates to drug screening assays designed to identify compounds that regulate TRPM2 and thereby also function to modulate ADPR-mediated cell migration. The invention is based on the discovery that, 8Br-ADPR, which specifically inhibits activation of TRPM2, acts to inhibit ADPR-mediated cell migration.

### 1. INTRODUCTION

The US government has a paid up license in this invention and the right in limited circumstances to require the patent owner to license of others on reasonable terms as provided for by the terms of contract number RO1 AI-057996 awarded by National Institute of Allergy and Infectious Disease and contract number P50 DA11806 awarded by National Institutes of Health.

The present invention relates to methods and compositions for modulating ADPR-mediated migratory activity of cells through regulation of the TRPM2 cation channel. Such methods and compositions may be used for the treatment of disorders including, but not limited to, inflammation, ischemia, atherosclerosis, asthma, autoimmune disease, diabetes, arthritis, allergies, and transplant rejection. Such cells include, for example, neutrophils, lymphocytes, eosinophils, macrophages, monocytes and dendritic cells. The invention further relates to specific inhibition of TRPM2 by blocking the activity of ADPR. The invention also relates to drug screening assays designed to identify compounds that regulate TRPM2 and thereby also function to modulate ADPR-mediated cell migration. The invention is based on the discovery that, 8Br-ADPR, which specifically inhibits activation of TRPM2, acts to inhibit ADPR-mediated cell migration.

### 2. BACKGROUND OF INVENTION

Initiation and maintenance of immune responses are critically dependent on leukocyte migration to inflamed tissues and secondary lymphoid organs (Rot et al., 2004 Annu. Rev. Immunol. 22:891-928.). Indeed, the efficacy of immune responses depends upon successful recruitment of phagocytes and precursor dendritic cells (DCs) to sites of inflammation, the trafficking of maturing DCs from the inflammatory site to the draining lymph node and the subsequent migration of effector lymphocytes back to the site of injury or infection. Likewise, potentially damaging immune responses can be maintained in a chronic fashion by the continued recruitment of leukocytes to inflamed tissues (Cravens et al., 2002 Immunol. Cell. Biol. 80:497-505). Motile cells, including lymphocytes, DCs and neutrophils, can sense the presence of exogenous and/or endogenous chemokines and chemoattractants produced in secondary lymphoid tissues or at the site of inflammation and are able respond to increasing concentrations of these chemoattractants by polarizing and then migrating towards their source (Manes et al., 2005 Semin. Immunol. 17:77-86). While it is now clear that phospho-lipid kinases and phosphatases such as P13-K and PTEN play important roles in regulating cell polarity and chemotaxis (Ridley et al., 2003 Science 302:1704-1709), there is still much that is not understood about the biochemical events that control leukocyte migration. For example, there is still substantial controversy over the role that calcium signaling plays in regulating chemotactic responses.

Inositol trisphosphate (IP₃) is the best-known calcium-mobilizing second messenger and has been shown to play a critical role in signal transduction in essentially all cell types that have been examined, including leukocytes (Berridge, M. J., 2005 Annu. Rev. Physiol. 67:1-21). IP₃, which is produced by Phopholipase C (PLC), induces intracellular calcium release from IP₃ receptor (IP₃R)-gated stores in the endoplasmic reticulum (Berridge, M. J., 2005 Annu. Rev. Physiol. 67:1-21). A number of groups have demonstrated that leukocytes lacking various PLC isoforms make defective calcium responses after chemokine receptor activation, yet appear to be competent to migrate in response to chemokines (Wu et al., 2000 J. Cell. Sci. 113(Pt 17):2935-2940; Jiang et al., 1997 Proc. Natl. Acad. Sci. U.S.A. 94:7971-7975). Thus, it was largely concluded that IP₃-induced calcium release is not required for chemotactic responses. However, there are numerous examples in the literature showing that either intracellular calcium release, extracellular calcium influx or a combination of both is necessary for the cytoskeletal and cellular morphological transformations necessary for directional cell migration (Pettit et al., 1998 Physiol. Rev. 78:949-967), Thus, it has been difficult to assess the true importance of calcium signaling in chemotactic responses.

Interestingly, over the last decade three novel calcium-mobilizing metabolites were identified and all of these metabolites can be produced by the ecto-enzyme CD38 (Schuber et al., 2004 Curr. Mol. Med. 4:249-261). CD38, which is expressed by most hematopoietic cells (Mehta et al., 1996 FASEB J. 10:1408-1417; Lund et al., 1998 Immunol. Rev. 161:79-93), catalyzes an ADP-ribosyl cyclase reaction to generate cyclic adenosine diphosphate ribose (cADPR) from its substrate nicotinamide adenine dinucleotide (NAD) (Howard et al., 1993 Science 262:1056-1059). CD38 can also catalyze a NAD glycohydrolase reaction to produce adenosine diphosphate ribose (ADPR) (Howard et al., 1993 Science 262:1056-1059) and a base-exchange reaction to produce nicotinic acid adenine dinucleotide phosphate (NAADP⁺) (Aarhus et al., 1995 J. Biol. Chem. 270:30327-30333). Cyclic ADP-ribose induces intracellular Ca²⁺ release from ryanodine receptor-dependent Ca²⁺ stores (Meszaros et al., 1993 Nature 354:76-78) in wide variety of cell types isolated from plants, animals, and protists (Lee, H. C. 2004 Curr. Mol. Med. 4:227-237). Likewise, NAADP⁺ induces calcium release from intracellular ryanodine receptor-gated stores (Langhorst et al., 2004 Cell Signal 16:1283-1289; Hohenegger et al., 2002 Biochem. J. 367:423-431) and regulates Ca²⁺ release in multiple cell types including T cells (Lee, H. C. 2004 Curr. Mol. Med. 4:227-237; Berg et al., 2000 J. Cell Biol. 150:581-588). ADPR in turn, was found to induce Ca²⁺ influx in myeloid cells by binding to the Nudix domain of a transient receptor potential nonselective cation channel, designated melastatin-related 2 (TRPM2) (Perraud et al., 2001 Nature 411:595-599; Hara et al., 2002 Mol. Cell. 9:163-173; Sano et al., 2001 Science 293:1327-1330). Experimental evidence from several labs now suggests that ADPR-dependent TRPM2 activation represents a cellular sensor for oxidative stress and may play an important role in regulating oxidant-induced cell death (Kuhn et al., 2005 Pflugers. Arch. 451:212-219). Interestingly, data from the Penner laboratory has shown that while ADPR at relatively high concentrations directly activates TRPM2, TRPM2 can be efficiently activated by very low concentrations of ADPR when cADPR is also present (Kolisek et al., 2005 Mol. Cell. 18:61-69). Likewise, it is now known that cADPR interacts with NAADP⁺ and IP₃ to mold the global calcium response (Morgan et al., 2002. Kluwer, Boston. 167-198, Gallione et al., 2005 Cell Calcium 38:273-280). Thus, it is becoming increasingly clear that there is extensive “cross-talk” between the individual calcium-mobilizing metabolites and the calcium stores that they regulate.

Whether the calcium-mobilizing metabolites produced by CD38 regulate leukocyte migration has been addressed. CD38 expression on neutrophils, monocytes and myeloid-derived DCs is required for the chemotaxis of these cells to several different chemokines and chemoattractants including bacterially-derived formylated peptides (fMLP) (Partida-Sanchez et al., 2001 Nature Medicine 7:1209-1216; Partida-Sanchez et al., 2004 Immunity 20:279-291; Partida-Sanchez et al., 2004 J. Immunol. 172:1896-1906). See also, U.S. patent application Ser. No. 11/115,964 which is incorporated herein by reference and which discloses the role of CD38 protein in chemotaxis. Moreover, migration of neutrophils and myeloid DC precursors to sites of inflammation as well as the migration of mature DCs from the site of inflammation to the draining lymph node is impaired in CD38 deficient (CD38KO) mice (Partida-Sanchez et al., 2001 Nature Medicine 7:1209-1216; Partida-Sanchez et al., 2004 Immunity 20:279-291). Consequently, these mice make poor innate and adaptive immune responses (Partida-Sanchez et al., 2001 Nature Medicine 7:1209-1216; Partida-Sanchez et al., 2004 Immunity 20:279-291; Cockayne et al., 1998 Blood 92:1324-1333). Consistent with the defective chemotaxis of DCs and neutrophils, CD38 deficient cells make impaired calcium responses to several chemokines (Partida-Sanchez et al., 2001 Nature Medicine 7:1209-1216; Partida-Sanchez et al., 2004 Immunity 20:279-291). For example, both intracellular calcium release and extracellular calcium influx were reduced in fMLP-stimulated CD38 deficient neutrophils (Partida-Sanchez et al., 2001 Nature Medicine 7:1209-1216; Partida-Sanchez et al., 2003 Microbes Infect. 5:49-58) and the calcium response was largely abrogated in CD38KO DCs stimulated with CCR7 and CXCR4 ligands (Partida-Sanchez et al., 2004 Immunity 20:279-291). Interestingly, when normal mouse neutrophils, human neutrophils, human monocytes or mouse DCs were pretreated with a cADPR antagonist, these cells also made defective calcium and chemotactic responses to a variety of different chemokines (Partida-Sanchez et al., 2001 Nature Medicine 7:1209-1216; Partida-Sanchez et al., 2004 Immunity 20:279-291; Partida-Sanchez et al., 2004 J. Immunol. 172:1896-1906). Therefore, based on these data, it was thought that CD38, through its production of cADPR, regulated calcium and chemotactic responses in human and mouse leukocytes (Schuber et al., 2004 Curr. Mol. Med. 4:249-261; Partida-Sanchez et al., 2003 Microbes Infect. 5:49-58).

Although these experiments indicate that cADPR, produced by CD38, regulates calcium signaling and chemotactic responses in leukocytes, they do not address the possibility that the other calcium-mobilizing metabolites produced by CD38 were also involved in this process. In fact, the most striking defect in the chemokine-treated CD38KO cells was the reduction in extracellular calcium influx seen in these cells (Partida-Sanchez et al., 2001 Nature Medicine 7:1209-1216; Partida-Sanchez et al., 2004 Immunity 20:279-291). ADPR, one of the metabolites produced by CD38 as well as by other enzymes including PARP-1 and PARG, is reported to activate TRPM2-mediated calcium influx alone and in combination with cADPR (Kolisek et al., 2005 Mol Cell 18:61-69). The present invention provides evidence of a role of ADPR in regulating calcium responses in chemokine-stimulated cells.

### 3. SUMMARY OF THE INVENTION

The present invention relates to methods and compositions for modulating the ADPR-mediated migratory activity of cells through regulation of the TRPM2 cation channel. Such methods and compositions may be used for the treatment of disorders including, but not limited to, inflammation, ischemia, atherosclerosis, asthma, autoimmune disease, diabetes, arthritis, allergies, and transplant rejection. Such cells include, for example, neutrophils, lymphocytes, eosinophils, macrophages, monocytes and dendritic cells. The invention further relates to specific inhibition of TRPM2 by blocking the activity of ADPR. The invention also relates to drug screening assays designed to identify compounds that regulate TRPM2 and thereby also function to modulate CD38 mediated cell migration. The invention is based on the discovery that, 8Br-ADPR, which specifically inhibits ADPR-gated calcium influx through TRPM2 or other ADPR-gated plasma membrane cation channels, acts to inhibit cell migration.

### 4. BRIEF DESCRIPTION OF THE FIGURES

FIG. 1. DCs and neutrophils express cation channels with the pharmacologic properties of TRPM2. PCR analysis using a primer pair specific for TRPM2 was performed on cDNA prepared from the purified neutrophils and DCs. The 589 bp TRPM2-specific product was detected in both neutrophils (PMN) and DCs after 37 amplification cycles.

FIG. 2. Synthesis of ADPR and cADPR analogues. A. Diagram of the scheme used to prepare 8Br-ADPR and 8Br-cADPR from 8Br-NAD⁺. B. HPLC profile of the purified compounds. C. HPLC profiles of the purified 8Br-ADPR before and 15 minutes after incubation with mouse bone marrow neutrophils. The HPLC profiles and relative percentages of 8Br-cADPR, 8Br-ADPR and 8Br-AMP present in the supernatants are shown and the HPLC profiles of standards for each of the compounds are included for comparison.

FIG. 3. 8Br-ADPR inhibits ADPR-gated Ca²⁺ influx, but not store-operated Ca²⁺ influx, in TRPM2-expressing leukocytes. (A) PCR analysis for TRPM2 was performed on cDNA prepared from mouse bone marrow neutrophils and immature DCs. The 589 bp TRPM2-specific product was detected in both neutrophils (PMN) and DCs after 35 amplification cycles but was not present in the no template control (Ø). (B-C) 8Br-ADPR blocks ADPR-gated cation entry in TRPM2-expressing cells. Panel B shows average membrane currents (±SEM) recorded at −60 mV in patch-clamped TRPM2-expressing Jurkat T cells infused with a vehicle control (n=14 cells, black), 300 μM ADPR alone (n=9 cells, red) or ADPR in combination with 900 μM 8Br-ADPR (n=16 cells, green). Panel C represents the maximal membrane current in Jurkat T cells infused with vehicle control, ADPR alone or ADPR+8Br-ADPR. (D) 8Br-ADPR does not inhibit store-operated Ca²⁺ entry in neutrophils or DCs. Mouse bone marrow-derived neutrophils (left), immature DCs (middle) and TNFα-matured DCs (right) were loaded with Fluo-3 and Fura-red and then preincubated for 15 minutes with media (black), or 100 μM 8Br-ADPR (green). Cells were then stimulated with thapsigargin (1 μM). Flow cytometry was used to measure the accumulation of intracellular free Ca²⁺. All data are representative of three or more independent experiments.

FIG. 4. 8Br-ADPR inhibits Ca²⁺ influx in chemoattractant-activated neutrophils and DCs. (A-D) Mouse bone marrow neutrophils were loaded with Fluo-3 and Fura-red and pre-incubated for 15 minutes in media (black), 8Br-cADPR (100 μM, blue) or 8Br-ADPR (100 μM, green). Cells were stimulated with fMLP (1 μM, panels A and C) or IL-8 (100 nM, panels B and D) and intracellular Ca²⁺ levels were measured by flow cytometry. In panels A and B, the extracellular Ca²⁺ was chelated with EGTA (2 mM) immediately before stimulation. (E) Immature bone-marrow derived DCs were sort-purified, loaded with Fluo-3 and Fura-red and pretreated for 15 minutes in media (black), 8Br-cADPR (100 μM, blue) or 8Br-ADPR (100 μM, green). The cells were then stimulated with CXCL12 (50 ng/ml) and intracellular free Ca²⁺ levels were measured by flow cytometry. The data shown are representative of 3 or more independent experiments.

FIG. 4F. 8Br-AMP does not block Ca²⁺ influx in chemokine stimulated neutrophils. WT bone marrow neutrophils were loaded with Fluo-3 and Fura-red and then preincubated in media (black) or 8Br-AMP (100 μM, red) for 15 minutes. The cells were stimulated with fMLP (1 μM). The accumulation of intracellular free Ca²⁺ was measured by flow cytometry. The data are representative of three independent experiments.

FIG. 5. 8Br-ADPR inhibits chemotaxis of mouse and human neutrophils and DCs to multiple chemoattractants. (A-B) Bone marrow-derived TNFα-matured DCs (A) and immature DCs (B) were sort-purified, pre-incubated for 15 minutes in media (black), 8Br-cADPR (blue, 100 μM) or 8Br-ADPR (green, 100 μM) and then placed in transwell chambers containing CCL21 (A) or CXCL12 (B) in the bottom chamber. The cells that migrated to the bottom chamber in response to the chemotactic gradient were collected and enumerated by FACS. The results are expressed as the mean ±SEM of the chemotaxis index (CI, see methods for description) of triplicate cultures. *p≦0.001 or **p≦0.015 between untreated DCs and all other groups. (C-D) Mouse bone marrow neutrophils were pre-incubated with media (black), 8Br-cADPR (100 μM, blue) or 8Br-ADPR (100 μM, green) and then placed in transwells containing 100 nM IL-8 (panel C) or 1 μM fMLP (panel D) in the bottom chamber. The cells that migrated in response to the chemotactic gradient were collected at 45 minutes and enumerated by flow cytometry. The data are reported as the mean ±SD of the CI of triplicate cultures. *p≦0.001 between untreated neutrophils and all other groups. (E) Mouse bone marrow neutrophils were incubated in the presence of increasing amounts of 8Br-ADPR (0-100 μM) for 15 minutes. The chemotactic response of the cells to fMLP (1 μM) was then determined as described above. The data are reported as the mean ±SD of the CI of triplicate cultures. *p≦0.003 between untreated neutrophils and all other groups. (F) Human peripheral blood neutrophils were pre-incubated in the presence of media (black), 8Br-cADPR (blue, 100 μM) or 8Br-ADPR (green, 100 μM) for 15 minutes. The chemotactic response of the cells to the FPRL1 ligand, A5 peptide (1 μM), was measured as described above. The results are expressed as the mean ±SD of the CI of triplicate cultures. *p<0.0001 or **p<0.001 between untreated neutrophils and all other groups. All data are representative of at least 4 independent experiments.

FIG. 6. CD38-expressing neutrophils convert 8Br-NAD into multiple metabolites that inhibit chemotactic responses. A-D. 8Br-NAD⁺ (panels A and B) or 8Br-cADPR (panels C and D) were incubated in the absence (0 min) or presence of purified WT (panels A and C) or CD38KO (panels B and D) bone marrow neutrophils for 3 to 15 min. The supernatant was collected from the centrifuged samples and then concentrated using 10 kDa MWCO centricon. The nucleotides present in the supernatants were then analyzed by HPLC. The relative percentage of 8Br-NAD, 8Br-ADPR, 8BR-cADPR and 8Br-AMP present in the cell lysates of cells treated with the brominated compounds is shown. E. After 15 min of incubation with 8Br-NAD, 8Br-ADPR or 8Br-cADPR, WT (open bars) or CD38KO (black bars) bone marrow neutrophils were placed in the top chamber of a transwell that contained 1 μM fMLP in the bottom chamber. The cells that migrated to the bottom chamber in 45 min in response to the chemotactic gradient were collected and enumerated by FACS. The data are reported as the mean ±SD of the CI of triplicate cultures. The data are representative of two independent experiments.

FIG. 7. Leukocyte chemotaxis is dependent on both ADPR and cADPR. (A) Purified 8Br-cADPR (100 μM) was incubated alone or in the presence of WT neutrophils for 15 minutes. The supernatants from the samples were collected and analyzed by HPLC to identify the brominated metabolites present in the cultures. The HPLC profiles of standards for each of the compounds are included for comparison and the relative proportion of each catabolite is indicated. (B) Mouse bone marrow neutrophils were incubated in the presence of increasing amounts of 8Br-cADPR (0-100 μM) for 15 minutes. The chemotactic response of the neutrophils to fMLP (1 μM) was then determined as described for FIG. 5. The data are reported as the mean ±SD of the CI of triplicate cultures. *p<0.04 between untreated neutrophils and indicated groups. The data are representative of two or more independent experiments.

FIG. 8. Differential regulation of Ca²⁺ signaling by CD38 and PARP-1. (A) Bone marrow neutrophils isolated from WT and Parp1^{-/-} mice were loaded with Fluo-3 and Fura-red and pre-incubated in media (WT black and Parp1^{-/-} dark blue) or 100 μM 8Br-ADPR (WT green and Parp1^{-/} red) for 15 minutes. The cells were stimulated with fMLP (1 μM) and intracellular free Ca²⁺ levels were measured by flow cytometry. (B) Parp1^{-/-} and WT bone marrow neutrophils were incubated in the presence or absence of 8Br-ADPR as described for panel A. The chemotactic response of the cells to 1 μM fMLP was measured as described for FIG. 5. The results are reported as the mean ±SD of the CI of triplicate cultures. No statistical difference between the CI of fMLP-stimulated WT and Parp1^{-/-} neutrophils. *p<0.01 comparing the CI of untreated to 8Br-ADPR treated groups. (C) WT and Cd38^{-/-} -bone marrow neutrophils were incubated in media (WT black and Cd38^{-/-} light blue) or 100 μM 8Br-ADPR (WT green and Cd38^{-/-} yellow) and the chemotactic response of the cells to 1 μM fMLP was measured as described for FIG. 5. The results are reported as the mean ±SD of the CI in triplicate cultures. P<0.0001 between untreated WT cells and all other groups. (D) Bone marrow neutrophils were loaded with Fluo-3 and Fura-red, pre-incubated in media (WT black, Cd38^{-/-} light blue, and Parp1^{-/-} dark blue) or 8Br-ADPR (100 μM, WT green and Cd38^{-/-} yellow) for 15 minutes and then exposed to H₂O₂ (100 μM). Flow cytometry was used to measure intracellular free Ca²⁺ levels. All data are representative of at least three independent experiments.

FIG. 9. A CD38 substrate analog blocks chemokine receptor signaling but not oxidant-induced Ca²⁺ influx. (A) 8Br-NAD⁺ (500 μM) was incubated in the absence (0 min) or presence of purified WT (black) or Cd38^{-/-} (light blue) bone marrow neutrophils. The supernatants from the samples were collected between 2-15 minutes and analyzed by HPLC to identify the metabolites present in the cultures. The average relative percentage of 8Br-ADPR present in the supernatants of duplicate cultures at a time 0, 2 and 15 minutes is shown. (B) WT or Parp1^{-/-} bone marrow neutrophils were pre-incubated in the presence of media (WT black and Parp1^{-/-} blue) or 100 μM 8Br-NAD⁺ (WT green and Parp1^{-/-} red) for 15 minutes. The results are expressed as the mean ±SD of the CI of triplicate cultures. (C-D) WT or Parp1^{-/-} bone marrow neutrophils were loaded with Fluo-3 and Fura-red and then pre-incubated in media or 100 μM 8Br-NAD⁺ for 15 minutes. The cells were stimulated with 1 μM fMLP (panel C) or 100 μM H₂O₂ (panel D) and intracellular free Ca²⁺ levels were determined. The data are representative of at least three independent experiments.

### 5. DETAILED DESCRIPTION OF THE INVENTION

The present invention relates to methods for regulating the ADPR-mediated migratory activity of cells involving the regulation of the ADPR-gated TRPM2 channel. The invention is based on the discovery that a specific inhibitor of TRPM2, 8BR-ADPR, inhibits cell migration. The present invention encompasses screening assays designed for the identification of modulators, such as agonists and antagonists, of TRPM2 channel activity which are also modulators of chemotaxis. The invention further relates to the use of such modulators in the treatment of disorders based on the ADPR-controlled migratory activity of cells to chemoattractants and inflammatory products. Such disorders include, but are not limited to, inflammation, ischemia, autoimmune disease, asthma, diabetes, arthritis, allergies, infections and organ transplant rejection.

### 5.1. Screening Assays

In accordance with the invention, a cell based assay system can be used to screen for compounds that modulate the activity of TRPM2 and thereby, modulate the chemoattractant induced Ca²⁺ influx and the migration of hematopoietic cells. To this end, cells that endogenously express TRPM2 can be used to screen for compounds. Such cells may also express CD38, including, for example, neutrophils, lymphocytes, eosinophils, macrophages, monocytes and dendritic cells. Alternatively, cell lines, such as 293 cells, COS cells, CHO cells, Thp-1 cells, fibroblasts, and the like, endogenously expressing TRPM2 or genetically engineered to express TRPM2 can be used for screening purposes. For screens utilizing host cells genetically engineered to express a functional TRPM2 protein, it would be preferred to use host cells that are capable of responding to chemoattractants or inflammatory stimuli. Further, oocytes or liposomes engineered to express the TRPM2 protein may be used in assays developed to identify modulators of TRPM2 activity.

The present invention provides methods for identifying compounds that alter one or more of the channel activities of TRPM2, including but not limited to, induction of Ca²⁺ and Ca²⁺ mediated cell reactions. Specifically, compounds may be identified that promote TRPM2 channel activities, i.e., agonists, or compounds that inhibit TRPM2 channel activities, i.e., antagonists. Compounds that inhibit TRPM2 channel activities will be inhibitory for chemoattractant induced calcium responses and cell migration. Compounds that activate TRPM2 channel activity will enhance chemoattractant induced calcium responses and cell migration. Such compounds may be compounds that interact with TRPM2 thereby modulating channel activity, or compounds that compete/facilitate activator binding to TRPM2. In addition, compounds may be identified that regulate TRPM2 expression and thereby regulate the level of cation channel activity within a cell.

The present invention provides for methods for identifying a compound that activates the TRPM2 cation channel comprising (i) contacting a cell expressing TRPM2 with a test compound and measuring the level of TRPM2 activity; (ii) in a separate experiment, contacting a cell expressing TRPM2 protein with a placebo or vehicle control and measuring the level of TRPM2 activity where the conditions are essentially the same as in part (i), and then (iii) comparing the level of TRPM2 activity measured in part (i) with the level of TRPM2 activity in part (ii), wherein an increased level of TRPM2 activity in the presence of the test compound indicates that the test compound is a TRPM2 activator. In a further embodiment of the invention, the method may comprise the step of testing whether the identified activator increases ADPR-mediated activities, including, for example, cell migration.

The present invention also provides for methods for identifying a compound that inhibits the TRPM2 cation channel comprising (i) contacting a cell expressing TRPM2 with a test compound and a known activator of the TRPM2 cation channel (i.e. ADPR or a chemoattractant) and measuring the level of TRPM2 activity; (ii) in a separate experiment, contacting a cell expressing TRPM2 with a placebo or vehicle control and an activator of the TRPM2 cation channel (i.e. ADPR or a chemoattractant), where the conditions are essentially the same as in part (i) and then (iii) comparing the level of TRPM2 activity measured in part (i) with the level of TRPM2 activity in part (ii), wherein a decrease level of TRPM2 activity in the presence of the test compound indicates that the test compound is a TRPM2 inhibitor. In a further embodiment of the invention, the method may comprise the step of testing whether the identified inhibitor decreases ADPR-mediated activities, including, for example, cell migration.

Depending on the assays used to detect TRPM2 activity, the methods described above for identifying activators and inhibitors of TRPM2 may utilize cells that also express CD38. Additionally, the assays may be done in the presence or absence of a chemoattractant in steps (i) and (ii). A “chemoattractant”, as defined herein, is a compound or molecular complex that induces the directional migration of cells via a mechanism that is dependent on calcium influx. An example of such a chemoattractant includes, but is not limited to, fMet-leu-Phe (fMLP). Other chemoattractants that may be used include, eotaxin, GRO-1, IP-10, SDF-1, BLC, Rantes, MIP-1α, MCP-3, MIP3α; IL-8, SLC, ELC, Lymphotactin, PAF, Ltb4, complement c5a, MCP-1, amyloid 13 peptide, serum amyloid A and histamine.

In utilizing the cell systems described above, the cells expressing the TRPM2 protein are exposed to a test compound or to vehicle controls e.g., placebos): After exposure, the cells can be assayed to measure the activity of TRPM2 or the activity of the CD38 mediated signal transduction pathway itself can be assayed.

The ability of a test molecule to modulate the activity of TRPM2 maybe measured using standard biochemical and physiological techniques. Responses such as activation or suppression of TRPM2 may be assayed utilizing cell based calcium and/or migration assays to identify compounds that are capable of inhibiting or activating chemoattractant induced ADPR-dependent calcium responses and cell migration. In non-limiting embodiments of the invention, changes in intracellular Ca²⁺ levels may be monitored through the use of calcium indicator dyes including, but not limited to, Indo, Fluo-3, Fluo-4, Fluo-5F, Fluo-4FF, Fluo-5N, Fura-Red, calcium green, calcium orange, calcium crimson, magnesium green, Oregon green, and Rhod-2. Further, changes in membrane potential resulting from modulation of the TRPM2 channel activity can be measured using a voltage clamp or patch recording methods. Directed migration of cells may also be monitored by standard chemotaxis assays in modified Boyden chambers or on slides. Such assay systems are described in further detail in the working example of the present specification (See, Example 6).

After exposure to the test compound, or in the presence of a test compound, cells can be stimulated with a chemoattractant such as fMLP and changes in intracellular calcium levels and/or cell migration may be measured. These measurements will be compared to cells treated with the vehicle control. Increased levels of intracellular Ca²⁺, increased Ca²⁺ entry, increased production of ADPR, increases in migration of cells toward a chemoattractant in the presence of a test compound indicates that the compound acts as an agonist to increase the Ca²⁺ response and increase chemoattractant-induced ADPR-dependent cell migration. Decreased levels of intracellular Ca²⁺, decreased Ca²⁺ entry and/or decreased migration of cells toward a chemoattractant in the presence of a test compound indicates that the compound acts as an antagonist and inhibits the Ca²⁺ response and inhibits chemoattractant induced ADPR-dependent cell migration.

In yet another embodiment of the invention, compounds that directly alter (i.e., activate or inactivate) the activity of ADPR, i.e., induced calcium influx and cell migration, can be tested in assays. Such agonists or antagonists would be expected to modulate the influx of Ca²⁺ into the cell resulting in changes in the cell's migratory activity. Antagonists would have reduced Ca²⁺ responses and/or reduced migration in the presence of a chemoattractant. Examples of antagonists include, but are not limited to 8-NH₂-ADPR, 8BR-ADPR, 8-CH₃-ADPR, 8-OCH3-ADPR 7-Deaza-8BR-ADPR and 8-azido-ADPR. Such a compound fitting these specifications is described in further detail in the working example of the present specification (Example 6). Agonists would have increased Ca²⁺ responses, and/or increased migration in the presence of chemoattractants. Examples of agonists include but are not limited to 2′-deoxy-ADPR, 3′-deoxy-ADPR and 2′-phospho-ADPR. Assays for direct measurement of APDR-gated calcium/cation influx activity include the bioassays such as those described by Sano et al (2001, Science 293:1327), Perraud et al (2001, Nature 411:595), Hara et al (2002, Molecular Cell 9:163) and Kolisek et al (2005, Molecular Cell 18:61)

Further, the assays of invention may identify compounds that are capable of activating the TRPM2 cation channel, i.e., agonists, but which cause desensitization of the chemoattractant receptor by depletion of intracellular calcium stores. Such desensitization may, in some instances, lead to inhibition of cell migration due to the depletion of calcium stores. Thus compounds may be identified that function as agonists in TRPM2-induced calcium influx assays but function as antagonists in chemotaxis assays. Such assays and compounds are within the scope of the present invention.

In practice, high throughput screens may be conducted using arrays of reactions. Such arrays may comprise at least one solid phase. Microtitre plates conveniently can be utilized as the solid phase. An anchored component is immobilized by non-covalent or covalent attachments. The surfaces may be prepared in advance and stored. In order to conduct the assay, the non-immobilized component is added to the coated surfaces containing the anchored component. After the reaction is completed, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the solid surface; e.g., using a labeled antibody specific for the previously non-immobilized component.

In accordance with the invention, a cell based assay system can be used to screen for compounds that modulate the expression of TRPM2 within a cell. Assays may be designed to screen for compounds that regulate TRPM2 expression at either the transcriptional or translational level. In one embodiment, DNA encoding a reporter molecule can be linked to a regulatory element of the TRPM2 gene and used in appropriate intact cells, cell extracts or lysates to identify compounds that modulate TRPM2 gene expression. Such reporter genes may include but are not limited to chloramphenicol acetyltransferase (CAT), luciferase, β-glucuronidase (GUS), growth hormone, or placental alkaline phosphatase (SEAP). Such constructs are introduced into cells thereby providing a recombinant cell useful for screening assays designed to identify modulators of TRPM2 gene expression.

Following exposure of the cells to the test compound; the level of reporter gene expression may be quantitated to determine the test compound's ability to regulate TRPM2 expression. Alkaline phosphatase-assays are particularly useful in the practice of the invention as the enzyme is secreted from the cell. Therefore, tissue culture supernatant may be assayed for secreted alkaline phosphatase. In addition, alkaline phosphatase activity may be measured by colorimetric, bioluminescent or chemiluminescent assays such as those described in Bronstein, I. et al. (1994, Biotechniques 17: 172-177). Such assays provide a simple, sensitive easily automatable detection system for pharmaceutical screening.

To identify compounds that regulate TRPM2 translation, cells or in vitro cell lysates containing TRPM2 transcripts may be tested for modulation of TRPM2 mRNA translation. To assay for inhibitors of TRPM2 translation, test compounds are assayed for their ability to modulate the translation of TRPM2 mRNA in in vitro translation extracts.

In an embodiment of the invention, the level of TRPM2 expression can be modulated using antisense, ribozyme, or RNAi approaches to inhibit or prevent translation of TRPM2 mRNA transcripts or triple helix approaches to inhibit transcription of the TRPM2 gene. Antisense and RNAi approaches involve the design of oligonucleotides (either DNA or RNA) that are complementary to TRPM2 mRNA. The antisense or RNAi oligonucleotides will be targeted to the complementary mRNA transcripts and prevent translation. Absolute complementarity, although preferred, is not required. One skilled in the art can ascertain a tolerable degree of mismatch by use of standard procedures to determine the melting point of the hybridized complex.

In an embodiment of the invention, the level of TRPM2 expression can be modulated using antisense, ribozyme, or RNAi approaches to inhibit or prevent translation of TRPM2 mRNA transcripts or triple helix approaches to inhibit transcription of the genes. Such approaches may be utilized to treat disorders such as inflammation, autoimmunity, atherosclerosis, asthma, diabetes and allergies where inhibition of TRPM2 expression is designed to prevent hematopoietically-derived cell migration. Antisense and RNAi approaches involve the design of oligonucleotides (either DNA or RNA) that are complementary to TRPM2 mRNA. The antisense or siNA oligonucleotides will be targeted to the complementary mRNA transcripts and prevent translation. Absolute complementarity, although preferred, is not required. One skilled in the art can ascertain a tolerable degree of mismatch by use of standard procedures to determine the melting point of the hybridized complex.

In a preferred embodiment of the invention, double-stranded short interfering nucleic acid (siNA) molecules may be designed to inhibit TRPM2 expression. In one embodiment, the invention features a double-stranded siNA molecule that down-regulates expression of the TRPM2 gene, wherein said siNA molecule comprises about 15 to about 28 base pairs.

In one embodiment, the invention features a double stranded short interfering nucleic acid (siNA) molecule that directs cleavage of a TRPM2 RNA via RNA interference (RNAi), wherein the double stranded siNA molecule comprises a first and a second strand, each strand of the siNA molecule is about 18 to about 28 nucleotides in length, the first strand of the siNA molecule comprises nucleotide sequence having sufficient complementarity to the TRPM2 RNA for the siNA molecule to direct cleavage of the TRPM2 RNA via RNA interference, and the second strand of said siNA molecule comprises nucleotide sequence that is complementary to the first strand.

In one embodiment, the invention features a double stranded short interfering nucleic acid (siNA) molecule that directs cleavage of a TRPM2 RNA via RNA interference (RNAi), wherein the double stranded siNA molecule comprises a first and a second strand, each strand of the siNA molecule is about 18 to about 23 nucleotides in length, the first strand of the siNA molecule comprises nucleotide sequence having sufficient complementarity to the TRPM2 RNA for the siNA molecule to direct cleavage of the TRPM2 RNA via RNA interference, and the second strand of said siNA molecule comprises nucleotide sequence that is complementary to the first strand.

In yet another embodiment of the invention, ribozyme molecules designed to catalytically cleave TRPM2 mRNA transcripts can also be used to prevent translation of TRPM2 mRNA and expression of TRPM2. (See, e.g., PCT International Publication WO90/11364, published Oct. 4, 1990; Sarver et al., 1990, Science 247:1222-1225). Alternatively, endogenous TRPM2 gene expression can be reduced by targeting deoxyribonucleotide sequences complementary to the regulatory region of the TRPM2 gene (i.e., the TRPM2 promoter and or enhancers) to form triple helical structures that prevent transcription of the TRPM2 gene in targeted cells in the body. (See generally, Helene, C. et al., 1991, Anticancer Drug Des. 6:569-584 and Maher, L J, 1992, Bioassays 14:807-815).

The oligonucleotides of the invention, i.e., antisense, ribozyme and triple helix forming oligonucleotides, may be synthesized by standard methods known in the art, e.g., by use of an automated DNA synthesizer (such as are commercially available from Biosearch, Applied Biosystems, etc.). Alternatively, recombinant expression vectors may be constructed to direct the expression of the oligonucleotides of the invention. Such vectors can be constructed by recombinant DNA technology methods standard in the art. In a specific embodiment, vectors such as viral vectors may be designed for gene therapy applications where the goal is in vivo expression of inhibitory oligonucleotides in targeted cells.

### 5.2. Compounds that can be Screened in Accordance with the Invention

Compounds which may be screened in accordance with the invention include, but are not limited to, small organic or inorganic compounds, peptides, antibodies and fragments thereof, and other organic compounds e.g., peptidomimetics) that bind to TRPM2 and either mimic the activity triggered by any of the known or unknown activators of TRPM2 (i.e., agonists) or inhibit the activity triggered by any of the known or unknown activators of TRPM2 (i.e., antagonists). Compounds that bind to TRPM2 and either enhance TRPM2 channel activities, i.e., agonists, or compounds that inhibit TRPM2 channel activities, i.e., antagonists, in the presence or absence of the chemoattractant will be identified. Compounds that bind to proteins that alter/modulate the channel activity of TRPM2 will be identified. Compounds that mimic natural activators, i.e., ADPR, can be identified. Compounds that directly activate or inhibit the ADPR-mediated Ca²⁺ signal transduction pathway in cells can be identified. Compounds that activate chemoattractant-induced ADPR-mediated calcium influx and chemotaxis will be identified. Compounds that inhibit chemoattractant-induced ADPR-mediated calcium influx and chemotaxis will be identified.

Compounds may include, but are not limited to, peptides such as, for example, soluble peptides, including but not limited to members of random peptide libraries (see, e.g., Lam, K. S. et al., 1991, Nature 354:82-84; Houghten, R. et al., 1991, Nature 354:84-86); and combinatorial chemistry-derived molecular library made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope binding fragments thereof), and small organic or inorganic molecules.

Other compounds which may be screened in accordance with the invention include but are not limited to small organic molecules that affect the expression of the TRPM2 gene or some other gene involved in the TRPM2 signal transduction pathway (e.g., by interacting with the regulatory region or transcription factors involved in gene expression); or such compounds that affect the cation channel activities of the TRPM2 or the activity of some other factor involved in modulating TRPM2 channel activity.

Additional compounds that may be screened also include compounds that are nucleotide and ADPR derivatives. In a specific embodiment of the invention, the ADPR backbone may be modified by, for example, combinatorial chemistry or by modifying the backbone with known adducts onto the adenosine and/or the ribose ring.

### 5.3. Compositions Containing Modulators of TRPM2 and their Uses

The present invention provides for methods of modulating cell migration comprising contacting a cell expressing TRPM2 with an effective amount of a TRPM2 modulating compound, such as a TRPM2 agonist or antagonist identified using the assays as set forth supra. Additionally, the present invention provides for methods of modulating TRPM2 mediated calcium responses and chemotaxis with an effective amount of a TRPM2 modulating compound, such as a TRPM2 agonist or antagonist identified using the assays as set forth supra. An “effective amount” of the TRPM2 inhibitor, i.e., antagonist, is an amount that decreases chemoattractant induced cell migration, decreases intracellular calcium levels, and/or that is associated with a detectable decrease in TRPM2 channel activity as measured by one of the above assays. An “effective amount” of the TRPM2 activator, i.e., agonist, is an amount that subjectively increases chemoattractant induced cell migration, increases intracellular calcium levels, and/or that is associated with a detectable increase in TRPM2 channel activity as measured by one of the above assays. Compositions of the invention also include modified TRPM2 activators, modulators of TRPM2 expression and agonists/antagonists of ADPR.

The present invention further provides methods of modulating cell migration in a subject, comprising administering to the subject, a composition comprising a compound that modulates TRPM2 channel activity identified as set forth in Section 5.1 supra. The composition may comprise an amount of TRPM2 channel activator or inhibitor, modulators of TRPM2 expression, modified TRPM2 substrates, or direct agonists/antagonists of ADPR controlled Ca2+ responses. Accordingly, the present invention provides for compositions comprising TRPM2 activators and inhibitors.

The invention provides for treatment or prevention of various diseases and disorders associated with cell migration by administration of a compound that regulates the expression or activity of TRPM2. Such compounds include but are not limited to TRPM2 antibodies; TRPM2 antisense nucleic acids, TRPM2 agonists and antagonists and ADPR agonists and antagonists. In a non-limiting embodiment of the invention, disorders associated with hematopoietic derived cell migration are treated or prevented by administration of a compound that regulates TRPM2 channel activity. Such disorders include but are not limited to inflammation, ischemia, atherosclerosis, asthma, auto-immune disease, diabetes, allergies, infections, arthritis and organ transplant rejections.

The present invention also provides pharmaceutical compositions. Such compositions comprise a therapeutically effective amount of a compound capable of regulating TRPM2 activity, ADPR activity or TRPM2 expression and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical sciences” by E. W. Martin. Such compositions will contain a therapeutically effective amount of the therapeutic compound, preferably in purified form, together with a suitable amount of carrier so as to provide the form for proper administration to the patient. The formulation should suit the mode of administration.

The compounds of the invention are preferably tested in vitro, and then in vivo for a desired therapeutic or prophylactic activity, prior to use in humans. For example, in vitro assays which can be used to determine whether administration of a specific therapeutic is indicated, include in vitro cell culture assays in which cells expressing TRPM2 are exposed to or otherwise administered a therapeutic compound and the effect of such a therapeutic upon TRPM2 activity is observed. In a specific embodiment of the invention the ability of a compound to regulate, i.e., activate or inhibit cell migration may be assayed.

Various delivery systems are known and can be used to administer a compound capable of regulating TRPM2 activity, ADPR activity, or TRPM2 expression, e.g., encapsulation in liposomes, microparticles, microcapsules, recombinant cells capable of expressing the compound, receptor-mediated endocytosis (see, e.g., Wu and Wu, 1987, J. Biol. Chem. 262:4429-4432). Methods of introduction include but are not limited to intradermal, intramuscular, intraperitoneal, intravenous, subcutaneous, intranasal, epidural, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. Pulmonary administration can also be employed, e.g., by use of an inhaler or nebulizer, and formulation with an aerosolizing agent.

In a specific embodiment, it may be desirable to administer the compositions of the invention locally to a specific area of the body; this may be achieved by, for example, and not by way of limitation, local infusion during surgery, topical application, e.g., in conjunction with a wound dressing after surgery, by patch, by injection, by means of a catheter, by means of a suppository, or by means of an implant, said implant being of a porous, non porous, or gelatinous material, including membranes, such as silastic membranes, or fibers.

The amount of the compound of the invention which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses maybe extrapolated from dose response curves derived from in vitro or animal model test systems. Additionally, the administration of the compound could be combined with other known efficacious drugs if the in vitro and in vivo studies indicate a synergistic or additive therapeutic effect when administered in combination.

The invention also provides a pharmaceutical pack or kit comprising one or more containers filled with one or more of the ingredients of the pharmaceutical compositions of the invention, optionally associated with such container(s) can be a notice in the form prescribed by a governmental agency regulating the manufacture, use or sale of pharmaceuticals or biological products, which notice reflects approval by the agency of manufacture, use or sale for human administration.

Various aspects of the invention are described in greater detail in the subsections below.

### 6. EXAMPLE

### ADP-Ribose Regulates Calcium Influx and Chemotaxis via TRPM-2 Channels in Neutrophils and Dendritic Cells

The subsection below describes data demonstrating that a novel compound, 8Br-ADPR blocks calcium influx through ADPR-gated TRPM2 cation channels. The data further show that ADPR, a product of the CD38 enzyme reaction, is required for calcium influx in chemoattractant-activated neutrophils and dendritic cells and that calcium influx is required for efficient migration of neutrophils and dendritic cells towards chemoattractants. The data show that inhibitors of TRPM2 or ADPR-gated calcium influx can be used to block cell migration.

### 6.1. Materials and Methods

Cell lines and mice. C57BL/6J (B6 mice), Cd38^{-/--} (N-B6.129P2-Cd38 tmlLnd mice backcrossed 12 generations to B6 (Cockayne, D. et al., 1998, Blood 92:1324-1333)), and Parp1^{-/-} mice (de Murcia, J. M. et al., 1997, Proc Natl Acad Sci USA 94:7303-7307; obtained from D. Chen at Lawrence Livermore laboratory and subsequently backcrossed 10 generations to B6) were bred and maintained at the Trudeau Institute Animal Breeding Facility in accordance with Trudeau Institute Institutional Animal Care and Use Committee guidelines. Jurkat T-Lymphocyte cells (clone JMP) were cultured and maintained as previously described (Gasser, A. et al., 2006, J Biol Chem 281:2489-2496).

Reagents. CXCL12 and CCL21 were acquired from R&D Systems, β-NAD⁺ was obtained from Roche Applied Science and the FPRL1 ligand, A5 peptide (Partida-Sanchez, S. et al., 2004 J Immunol 172:1896-1906), was purchased from New England Peptide. Trifluoroacetic acid (TFA) was from Pierce Biochemicals and AG MP-1 resin was from Bio-Rad. Human recombinant CD38 was a generous gift from Drs. H. C. Lee and R. Graeff (Dept. of Pharmacology, University of Minnesota). ADPR, IL-8, fMLP, H2O2, thapsigargin, EGTA, liquid Br2, tri-n-octylamine, 1,1,2-trichlorotrifluoroethane, Aplysia ADP-ribosyl cyclase were all obtained from Sigma-Aldrich. All reagents were used at the concentrations as indicated.

Detection of TRPM2 mRNA. PCR reactions were performed using TRPM2 specific primers (5′-TGCCTTTGGTGACATCGTTTTC-3′ and 5′-GATGGCCACACCTCCCCTTTCCTTC-3′) and cDNA prepared from mouse bone marrow neutrophils and DCs. A 589 bp TRPM2-specific product was detected after 35 cycles of amplification (30 sec at 94° C., 30 sec at 68° C., and 30 sec at 72° C.).

Synthesis of brominated compounds. The synthesis and purification of 8Br-NAD⁺ and 8Br-cADPR was performed as previously described (Walseth, T. F., 1993 Biochim Biophys Acta 1178:235-242). 8Br-ADPR was synthesized by incubating 8Br-NAD⁺ with human recombinant CD38 (0.1 μg/ml) for 2 hours at 25° C. 8Br-ADPR was then purified on a 1.6×11 cm AG MP-1 column. The 8Br-ADPR was eluted at 2.5 ml/min with a concave upward gradient of TFA from 1.5 to 150 mM over 32 minutes. 8Br-ADPR eluted between 22 to 29 minutes. To prevent breakdown of 8Br-ADPR, the TFA was extracted from the purified 8Br-ADPR by treating the pool (17.5 ml) with 12 ml of a 3:1 mixture of 1,1,2 trichlorotrifluoroethane/tri-N-octylamine (Khym, J. X., 1975 Clin Chem 21:1245-1252). Remaining acid was neutralized by adding 2M Tris-base and 1M NaOH to 1 and 2 mM, respectively and the sample was then dialyzed against distilled water. The purity of each of the brominated compounds was confirmed by analyzing 50 to 100 nmol of purified product on an analytical AG MP-1 column (0.5×5 cm). The preparations used were >95% pure.

Purification of neutrophils and DCs. Bone marrow neutrophils were purified by positive selection using biotinylated GR-1 (BD PharMingen) and MACS Streptavidin Microbeads (Miltenyi Biotec). Neutrophil purity was ≧95% as assessed by FACS. Human leukocytes were isolated from fresh peripheral blood and purified (95% purity) using a one-step Ficoll gradient (Robbins Scientific). Blood from normal healthy volunteers was provided by the Blood Donor Center, Champlain Valley Plattsburgh Hospital, Plattsburgh, N.Y. in accordance with the Trudeau Institute Institutional Review Board regulations. To isolate immature DCs, mouse bone marrow cells were cultured in complete media containing GMCSF (20 ng/ml) for 6-8 days and the CD11c⁺ClassII^{low} cells were sort-purified using a FACS Vantage SE with DiVa option (Becton Dickinson). To induce DC maturation, TNFα (10 ng/ml) was added to the cultures on day 6 and the mature CD 11 c⁺class-II^{hi} cells were sort-purified 48 hrs later.

Ca²⁺ mobilization assays. Bone marrow neutrophils (1×10⁷/ml) and DCs (1×10⁶/ml) were loaded with a mixture of Fluo-3 AM and Fura-Red AM as previously described (Partida-Sanchez, S. et al., 2001 Nature Medicine 7:1209-1216; Partida-Sanchez S. et al., 2004 Immunity 20:279-291). The cells were preincubated in media, 8Br-cADPR, 8Br-ADPR or 8Br-NAD⁺ (100 μM each) for 15 minutes and then stimulated. The accumulation of intracellular free Ca²⁺ was assessed by flow cytometry by measuring the fluorescence emission of Fluo-3 in the FL-1 channel and Fura-Red in the FL-3 channel over time. Data were analyzed using FlowJo 4.0 software (Tree Star). Relative intracellular free Ca²⁺ levels are expressed as the ratio between Fluo-3 and Fura-Red mean fluorescence intensity.

Chemotaxis assays. Chemotaxis assays were performed as previously described (Partida-Sanchez, S. et al., 2001 Nature Medicine 7:1209-1216; Partida-Sanchez S. et al., 2004 Immunity 20:279-291). Briefly, cells were pretreated for 15 minutes with media 8Br-cADPR, 8Br-ADPR or 8Br-NAD⁺ (100 μM each). Treated cells (1×10⁶ neutrophils or 1×10⁵ DCs) were added to the upper chamber of the transwell (3-μm for neutrophils or 5-μm for DCs) (Costar). After incubating the chambers for 45 min (neutrophils) or 90 min (DCs) at 37° C., the transmigrated cells were collected from the lower chamber, fixed, and counted on a flow cytometer. The results are expressed as the mean ±SD of the chemotaxis index (CI) for triplicate wells. The CI represents the fold-change in the number of untreated or inhibitor-pretreated cells that migrated in response to the chemoattractant divided by the basal migration of untreated or antagonist pretreated cells migrating in response to control medium.

Electrophysiology. Membrane currents were recorded in the whole-cell configuration of the patch-clamp technique (Hamill O. P. et al., 1981 Pflugers Arch 391:85-100). An EPC9 patch-clamp amplifier was used in conjunction with the PULSE stimulation and data acquisition software (HEKA Elektronik). The patch electrodes were made from 1.5 mm diameter borosilicate glass capillaries and filled with intracellular solution. Data were low-pass filtered at 1 kHz and compensated for both fast and slow capacity transients. Series resistance was compensated by 50-90%. All experiments were performed at room temperature with Jurkat T lymphocytes attached to high molecular weight poly-L-lysin. The pipette solution contained 145 mM K-glutamate, 8 mM NaCl, 1 mM MgCl₂ and 10 mM EGTA, adjusted to pH 7.2 with KOH and to a free Ca²⁺ concentration of 100 nM with CaCl₂. In some experiments, the pipette solution additionally contained ADPR (0.3 mM) or ADPR (0.3 mM) plus 8Br-ADPR (0.9 mM). The external solution contained 145 mM NaCl, 2 mM MgCl2, 1 mM CaCl2, 2.8 mM KCl, 10 mM HEPES and 10 mM glucose, adjusted to pH 7.2 with NaOH. The cells were held at −60 mV and I-V relations were obtained every 20 sec using 250 ms voltage ramps from −100 to +100 mV.

HPLC analysis of catabolites produced by CD38-expressing cells. Neutrophils were incubated with 8Br-NAD⁺ (500 μM), 8Br-cADPR (100 μM), or 8Br-ADPR (100 μM) for 0 (no cells in reaction) to 15 minutes at 37° C. The supernatants were collected after centrifugation, concentrated and flash frozen. Aliquots were analyzed by reversed-phase HPLC (Kontron Instruments) using a Multohyp BDS C18 column (250 mm×4.6 mm, particle size 5 μm, Chromatographie Service). Absorbance was measured at 270 nm using a UV detector (Kontron 432) and data were processed by the MT2 data acquisition system from Kontron Instruments. Peaks were identified by comparison to known standards and the area under each curve was quantified to determine relative amounts of each metabolite.

Statistical Analysis. Data sets were analyzed using GraphPad Prism version 4.0 for Macintosh (GraphPad Software). Student's t-test analyses were applied to the data sets and differences were considered significant when p values were ≦0.05.

### 6.2 Results

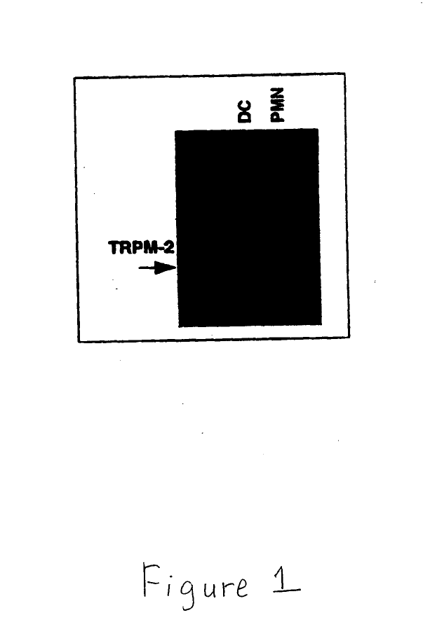

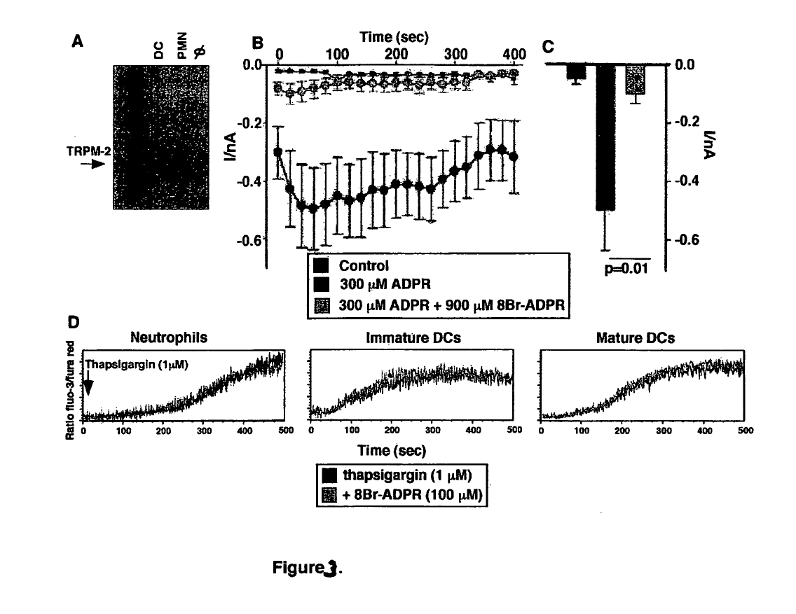

An ADPR analog blocks ADPR-gated cation entry in TRPM2-expressing cells. PCR was used to test whether TRPM2 transcripts were expressed by freshly isolated mouse bone marrow neutrophils and bone marrow-derived immature DCs. Similar to previous reports using human neutrophils (Heiner, I. et al., 2003 Cell Calcium 33:533-540) and a human monocyte cell line (Sano Y. et al., 2001, Science 293:1327-1330), it was found that mouse bone marrow neutrophils as well as mouse myeloid-derived DCs express TRPM2 mRNA (FIGS. 1A and 3A).

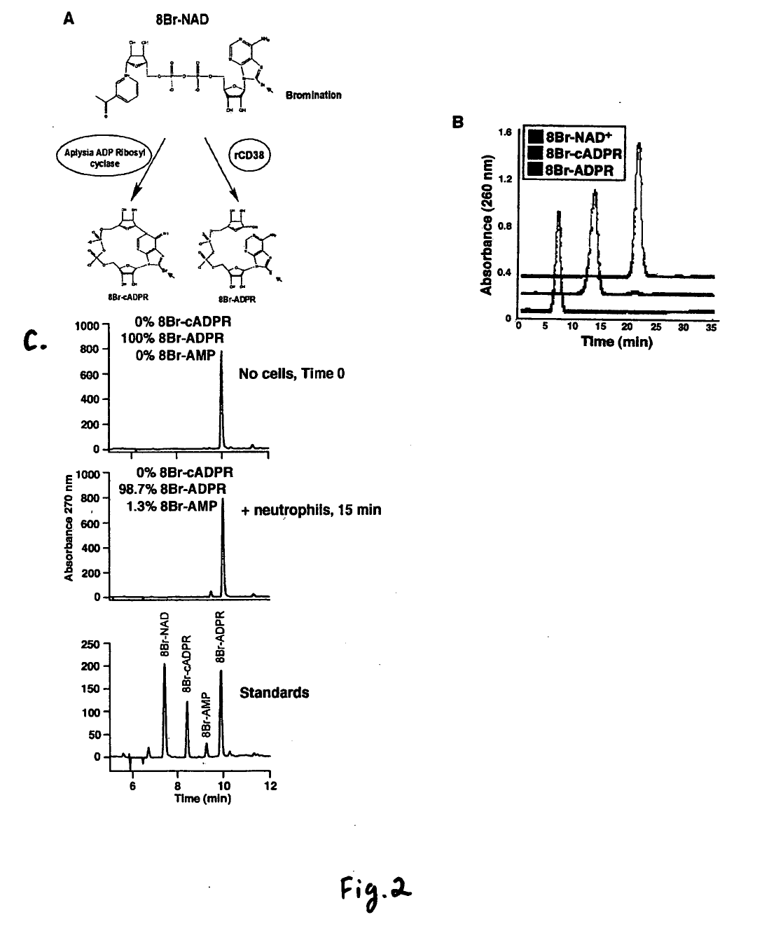

To date, no TRPM2-specific inhibitors have been identified (Kuhn, F. J. et al., 2005 Pflugers Arch 451:212-219), therefore, in order to assess the requirement for ADPR in chemotactic responses, the identification of a compound that could block ADPR-gated Ca2+ influx was needed. Previous work had shown that TRPM2 channels are activated by binding of ADPR to-the cytoplasmic NUDT9-H domain (reviewed in Miller, B. A., 2006 J Membr Biol 209:31-41). Based on these data, it was postulated that a brominated analog of ADPR might block ADPR-mediated activation of TRPM2. To test this hypothesis, 8-bromo adenosine diphosphoribose was synthesized and purified. (8Br-ADPR; FIG. 2A). HPLC analysis indicated that the compound was very pure and remained stable even after incubation with leukocytes, as <1.5% of the compound was catabolized to 8Br-AMP over 15 minutes (FIG. 2C). Importantly, no 8Br-cADPR was detected in the 8Br-ADPR preparation either before or after incubation with neutrophils (FIG. 2C).

To test whether 8Br-ADPR blocked ADPR-mediated cation entry through TRPM2 channels, a patch clamp analysis was performed on TRPM2-expressing Jurkat T cells (Gasser, A. et al., 2006 J Biol Chem 281:2489-2496; Beck, A. et al., 2006 Faseb J 25:1804-1815). Infusion of intracellular buffer alone (control) into the Jurkat cells had no effect, while infusion of ADPR in the pipette into individual Jurkat cells caused a slowly developing inward current across the membrane (FIG. 3B-C). The inward current was characterized by a linear I-V relationship typical for TRPM2 channels (data not shown). In contrast, when the pipette contained ADPR and a three-fold excess of 8Br-ADPR, the ADPR-induced cation entry was abrogated in the Jurkat cells (FIG. 3B-C), indicating that 8Br-ADPR blocks ADPR-gated Ca²⁺ influx in TRPM2-expressing cells.

In addition to TRPM2 channels, leukocytes also express store-operated Ca²⁺ channels (SOC) that are activated in response to intracellular Ca²⁺ store depletion (Ufret-Vincenty, C. A. et al., 1995 J Biol Chem 270:26790-26793). To test whether Ca²⁺ influx through SOCs is also inhibited by 8Br-ADPR, mouse neutrophils were incubated in the presence or absence of 8Br-ADPR and then stimulated the cells with thapsigargin, a drug that causes intracellular Ca²⁺ store depletion and subsequent Ca²⁺ entry through SOCs (Vostal, J. G. et al., 1996 J Biol Chem 271:19524-19529). Interestingly, 8Br-ADPR pretreatment of mouse bone marrow neutrophils and DCs had absolutely no effect on capacitative Ca²⁺ influx induced by thapsigargin (FIG. 3D). Taken together, these data indicate that 8Br-ADPR blocks ADPR-gated cation entry and does not block store-operated Ca²⁺ influx, indicating that 8Br-ADPR specifically inhibits ADPR-gated Ca²⁺ entry, presumably through TRPM2.

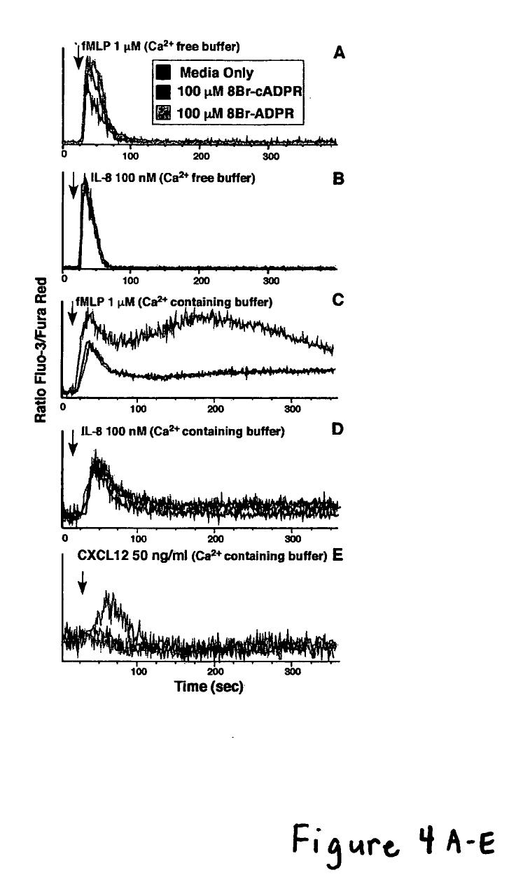

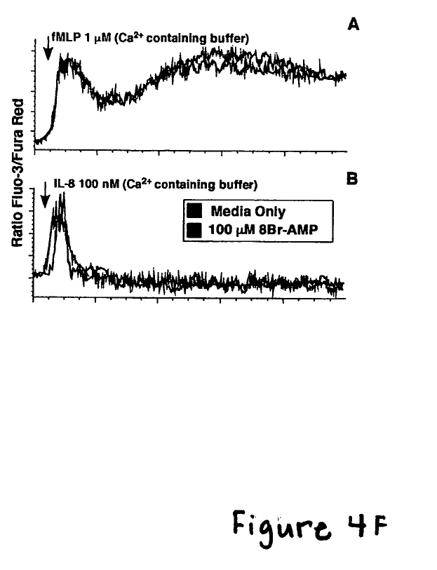

Ca2+ influx in chemoattractant-stimulated neutrophils and DCs is gated by ADPR. To determine whether the Ca²⁺ mobilization in chemokine-stimulated neutrophils and DCs is dependent on ADPR-gated Ca²⁺ influx bone marrow neutrophils were loaded with Ca²⁺ sensitive fluorescent dyes and pretreated the cells for 15 minutes with 8Br-ADPR or with the cADPR antagonist, 8Br-cADPR. Intracellular free Ca²⁺ levels were measured in cells stimulated with fMLP, a ligand for mFPR1, or with IL-8, a ligand for CXCR1 and CXCR2. To analyze the effect of 8Br-cADPR and 8Br-ADPR on Ca²⁺ mobilization from intracellular Ca²⁺ stores, experiments were first performed in Ca²⁺ free buffers. Consistent with results using Cd38^{-/-} neutrophils (Partida-Sanchez, S. et al., 2001 Nature Medicine 7:1209-1216), it was found that 8Br-cADPR pretreatment decreased intracellular Ca²⁺ release in the fMLP-stimulated neutrophils by approximately 25% (FIG. 4A) but had no effect on IL-8 induced intracellular Ca²⁺ release (FIG. 4B). In contrast, 8Br-ADPR treatment had no effect on intracellular Ca²⁺ release after either fMLP (FIG. 4A) or IL-8 stimulation (FIG. 4B).

To assess the potential role of ADPR in regulating extracellular Ca²⁺ influx in chemokine-stimulated leukocytes, the same experiments were performed in Ca²⁺-containing media. A biphasic Ca²⁺ response was observed in WT neutrophils stimulated with fMLP (FIG. 4C) that included a prolonged influx of extracellular Ca²⁺. It was found that the influx of extracellular Ca²⁺ was significantly decreased in WT neutrophils that were pre-treated with 8Br-cADPR (FIG. 4C). Interestingly, 8Br-ADPR pretreatment also caused a significant reduction in Ca²⁺ influx in the fMLP-stimulated neutrophils (FIG. 4C). Again neither compound had any effect on the IL-8 induced Ca²⁺ response (FIG. 4D). Importantly, the inhibition of Ca²⁺ influx observed in the 8Br-ADPR-treated, fMLP-stimulated neutrophils was specific, as pretreatment of the neutrophils with 8Br-AMP, the only other brominated nucleotide present (See FIG. 4E), had absolutely no effect on fMLP-induced Ca²⁺ influx.

To determine whether the effect of 8Br-ADPR on Ca²⁺ influx was limited to a single chemoattractant receptor or cell type, the effect of 8Br-ADPR on the Ca²⁺ response of mouse DCs that were stimulated with the CXCR4 ligand, CXCL12, was analyzed. This response was shown to be dependent on CD38, cADPR and Ca²⁺ influx (Partida-Sanchez, S. et al., 2004 Immunity 20:279-291). Therefore, sort-purified immature DCs from day 8 GMCSF-cultured bone marrow cells were loaded with Ca²⁺-sensitive dyes, preincubated for 15 minutes with media, 8Br-cADPR or 8Br-ADPR, and then stimulated the cells with CXCL12. Pretreatment of the DCs with 8Br-cADPR blocked the Ca²⁺ response of the CXCL12-stimulated DCs (FIG. 4E). Interestingly, 8Br-ADPR pretreatment also blocked CXCL12-induced Ca²⁺ responses (FIG. 4E). Similar results were observed when we treated purified mature splenic DCs with 8Br-ADPR and measured the Ca²⁺ response to the CCR7 ligands, CCL 19 or CCL21 (data not shown). Together, these data indicate that ADPR regulates extracellular Ca²⁺ influx in at least two distinct cell types activated with different chemoattractants.

8Br-AMP does not block Ca²⁺ influx in chemokine stimulated neutrophils. WT bone marrow neutrophils were loaded with Fluo-3 and Fura-red and then preincubated in media (black) or 8Br-AMP (100 μM, red) for 15 minutes. The cells were stimulated with fMLP (1 μM). The accumulation of intracellular free Ca²⁺ was measured by flow cytometry. The data presented in FIG. 4F are representative of three independent experiments.

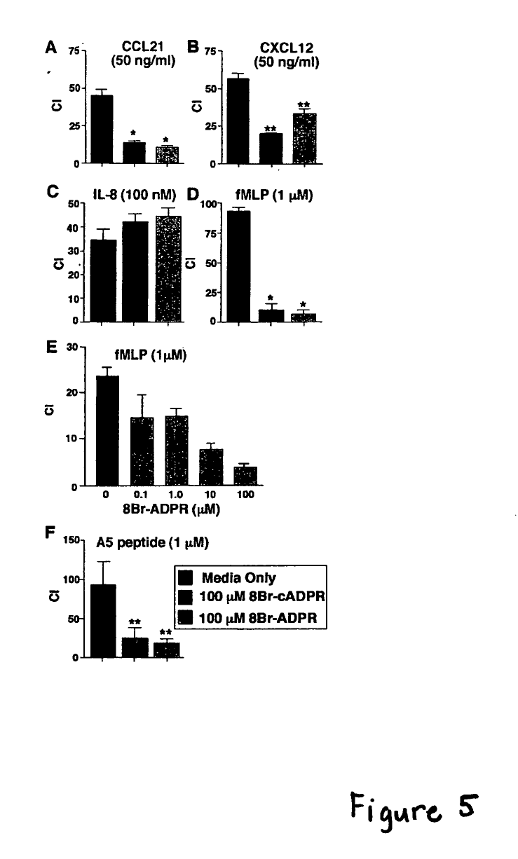

Chemotaxis of human and mouse leukocytes is dependent on ADPR-gated Ca²⁺ influx. It was previously demonstrated that chemotaxis of mouse bone marrow neutrophils and DCs to mFPR1, CXCR4 and CCR7 ligands is dependent on Ca²⁺ influx (Partida-Sanchez, S. et. al, 2001 Nature Medicine 7:1209-1216; Partida Sanchez, S. et al., 2004 Immunity 20:279-291). Since 8Br-ADPR blocked Ca²⁺ influx in the chemokine-stimulated DCs and neutrophils, it was predicted that 8Br-ADPR would also inhibit the chemotaxis of neutrophils and DCs to these chemoattractants. To test this hypothesis, bone marrow-derived immature or TNFα-matured DCs were pretreated with 8Br-cADPR or 8Br-ADPR and the chemotactic response of the cells to CXCL12 (immature DCs) or CCL21 (mature DCs) was measured. A very robust chemotactic response was observed from the untreated mature (FIG. 5A) and untreated immature DCs (FIG. 5B) to CCL21 and CXCL12, respectively. As previously reported (Partida Sanchez, S. et al., 2004 Immunity 20:279-291), neither the immature nor mature DCs migrated efficiently to CXCL12 or CCL21 when they were pretreated with 8Br-cADPR (FIG. 5A-B). Similarly, the 8Br-ADPR-treated immature and mature DCs made poor chemotactic responses to CXCL12 and CCL21 (FIG. 5A-B), indicating that ADPR-gated Ca²⁺ influx is required for the chemotaxis of DCs to CXCR4 and CCR7 ligands.

Next, mouse bone marrow neutrophils were pretreated with 8Br-cADPR or 8Br-ADPR and then the chemotactic response of these cells to fMLP or IL-8 was measured. Similar to the results measuring Ca²⁺ responses (FIG. 4), neither 8Br-ADPR nor 8Br-cADPR blocked the chemotactic response of the mouse neutrophils to IL-8 (FIG. 5C). Pretreating neutrophils with 8Br-cADPR effectively blocked chemotaxis (FIG. 5D). Likewise, the chemotactic response of the neutrophils to fMLP was efficiently inhibited by pretreatment with 8Br-ADPR (FIG. 5D). The inhibitory effect of 8Br-ADPR on neutrophil chemotaxis was very potent as treatment of cells with low micromolar concentrations of 8Br-ADPR (2.5 μM) was sufficient to inhibit cell migration by at least 50% (FIG. 5E). Thus, chemotaxis of mouse bone marrow neutrophils to mFPR1 ligands is dependent on ADPR-gated Ca²⁺ influx.

It was previously shown that chemotaxis of human neutrophils to FPRL1 ligands is dependent on cADPR and Ca²⁺ influx through a plasma membrane channel (Partida-Sanchez, S. et al., 2004 J. Immunol 172:1896-1906). To assess whether ADPR-mediated Ca²⁺ influx is required for the chemotaxis of human neutrophils to FPRL1 ligands, human neutrophils were purified from the peripheral blood of healthy volunteers and pretreated with 8Br-cADPR or 8Br-ADPR. The chemotactic response of these cells to A5 peptide, a specific ligand for human FPRL1 (Partida-Sanchez, S. et al., 2004 J. Immunol 172:1896-1906) was then measured. As shown in FIG. 5F, the untreated human neutrophils made a very significant chemotactic response to A5 peptide. In contrast, pretreatment with either 8Br-cADPR or 8Br-ADPR effectively inhibited the migration of these cells to the FPRL1 ligand (FIG. 5F). Taken altogether, these data demonstrate that ADPR-gated Ca²⁺ influx, presumably through TRPM2, is required for chemotaxis of human and mouse neutrophils and DCs to multiple, although not all, chemoattractants.

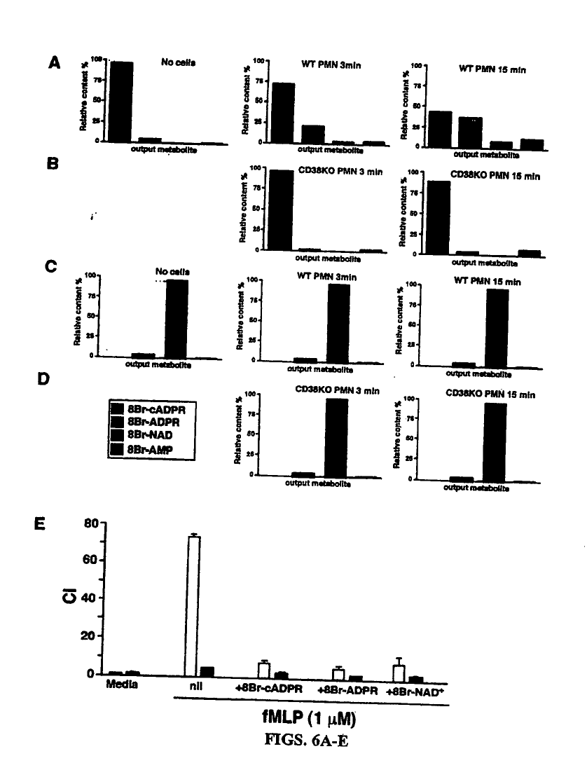

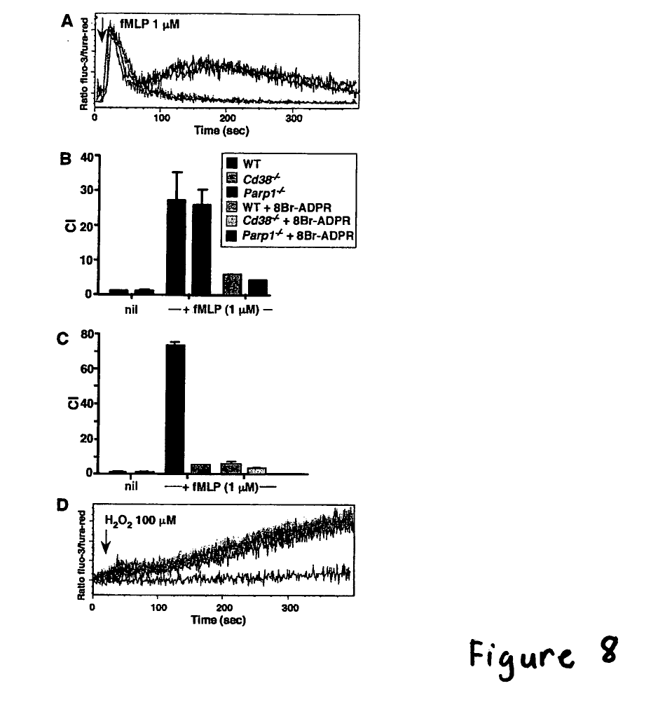

Both cADPR and ADPR are required to activate calcium influx in chemoattractant stimulated neutrophils and DCs. All together, the data suggested that ligation of a subset of chemokine receptors activates calcium influx through a plasma membrane cation channel that is regulated by both ADPR and cADPR, as activation of this channel was inhibited by cADPR as well as ADPR antagonists. However, most hematopoietic cells express the ecto-enzyme CD38 and this enzyme catalyzes the formation of cADPR and ADPR from its substrate NAD (Schuber, et al., 2004 Curr. Mol. Med. 4:249-261). Furthermore, it was reported that CD38 possesses cADPR hydrolase activity and can utilize cADPR as a substrate to produce ADPR (Howard, et al., 1993 Science 262:1056-1059). Although this reaction is highly inefficient (Schuber, et al., 2004 Curr. Mol. Med. 4:249-261), it was important to assess whether the CD38-expressing cells catabolized the cADPR antagonist, 8Br-cADPR, into the ADPR antagonist, 8Br-ADPR. To test this possibility, purified 8Br-cADPR or 8Br-NAD⁺ was incubated with CD38-expressing and CD38KO bone marrow neutrophils for 0-15 min and then HPLC analysis was used to determine the relative proportions of the catabolites present in the medium. As shown in FIG. 6, the 8Br-NAD⁺ (FIG. 6A) and 8Br-cADPR (FIG. 6C) remained intact when the compounds were incubated in the absence of any cells (time 0). However, when the WT neutrophils were incubated with 8Br-NAD⁺ for 3 to 15 minutes, the 8Br-NAD⁺ was rapidly converted into 8Br-ADPR and, to a smaller extent, into 8Br-cADPR (FIG. 6A). The catabolism of 8Br-NAD⁺ was CD38 dependent as neither 8Br-ADPR nor 8Br-cADPR were detected in the supernatants of the CD38KO cells that were incubated with 8Br-NAD⁺ (FIG. 6B). 8Br-AMP was detected in small quantities in the supernatants of both CD38KO and WT neutrophils incubated with 8Br-NAD⁺ (FIG. 6A-B), presumably due to degradation of the 8Br-NAD⁺ by an ecto-pyrophosphatase such as PC-1 (Goding, et al., 1998 Immunol. Rev. 161:11-26). In contrast, regardless of whether 8Br-cADPR was incubated with CD38-expressing cells (FIG. 6C) or CD38KO cells (FIG. 6D), it remained largely unchanged and no 8Br-ADPR was detected in either culture even after 15 minutes of incubation (FIG. 6C-D). Importantly, when the WT cells were cultured for 15 min in the presence of either 8Br-cADPR or 8Br-NAD⁺, the chemotactic response of the cells to fMLP was significantly inhibited (FIG. 6E) and was equivalent to that seen in CD38KO neutrophils. Furthermore, no further inhibition of the chemotactic response was observed in CD38KO neutrophils treated with 8Br-NAD⁺, 8Br-cADPR or 8Br-ADPR (FIG. 6E). Taken together, these data indicate that both ADPR and cADPR are necessary to activate the TRPM2 plasma membrane channel on leukocytes activated with chemoattractants like fMLP. In addition, the data suggest that CD38 regulates neutrophil and DC trafficking by producing cADPR and cADPR in combination with ADPR produced by CD38, or perhaps by another enzyme, are needed to activate TRPM2-mediated calcium influx and chemotaxis.

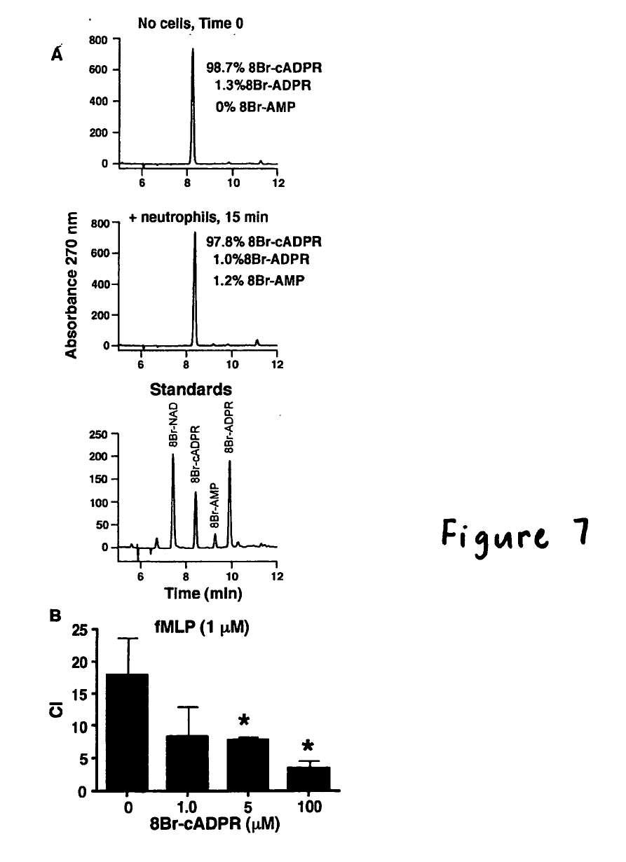

Chemotaxis and Ca²⁺ responses in chemokine-stimulated leukocytes are dependent on both cADPR and ADPR. Although cADPR was first identified as a Ca²⁺-signaling second messenger that mobilizes intracellular Ca²⁺ release (Lee, H. C., 2004 Curr Mol Med 4:227-237), data indicated that 8Br-cADPR also blocks extracellular Ca²⁺ influx. Given that cADPR can be hydrolyzed, albeit very inefficiently, to ADPR by CD38 (Howard, M. et al., 1993 Science 262:1056-1059), it was important to assess the stability of the 8Br-cADPR preparation to ensure that it was not degraded to 8Br-ADPR. Thus, purified 8Br-cADPR was incubated alone or with CD38-expressing bone marrow neutrophils for 15 minutes and the supernatant was analyzed by HPLC analysis to determine the relative proportions of the different brominated catabolites. The purity of the 8Br-cADPR used in these studies was very high (FIG. 7A, 98-99%). Furthermore, although a very small amount of 8Br-ADPR was detected in the preparation (<2% of the total compound), no significant additional hydrolysis was observed after the 15 minute incubation with CD38-expressing bone marrow neutrophils (FIG. 7A). However, to address whether the small amount of contaminating 8Br-ADPR was responsible for the inhibition of chemotaxis observed, mouse neutrophils were treated with increasing amounts of 8Br-cADPR and then chemotaxis of the treated cells to fMLP was measured. Similar to what was previously found with FPRL1-activated human neutrophils (Partida-Sanchez, S. et al., 2004 J. Immunol 172:1896-1906), it was determined that the IC₅₀ of 8Br-cADPR on fMLP-stimulated mouse neutrophils was in the low micromolar range (FIG. 7B, IC₅₀˜1-5 μM). Since the amount of contaminating 8Br-ADPR present in a 1 μM solution of 8Br-cADPR was <20 nM, a value well below the IC₅₀ of this compound (see FIG. 5E), the data support the conclusion that both cADPR and ADPR are required to activate Ca²⁺ influx in chemokine-stimulated TRPM2-expressing leukocytes.

Ca²⁺ entry in response to chemokines and oxidants is regulated by distinct mechanisms. Data indicates that cADPR and ADPR are each necessary for activation of Ca²⁺ influx in chemokine-stimulated leukocytes. Unlike cADPR, for which CD38 appears to be the major or even sole source in bone marrow neutrophils and DCs (Partida-Sanchez, S. et al., 2001 Nature Medicine 7:1209-1216; Partida-Sanchez, S. et al., Immunity 20:279-291), free ADPR can be produced by CD38 as well as by the PARP-1/PARG metabolic pathway. To test whether ADPR generated by the PARP-1/PARG pathway regulates chemoattractant-induced Ca²⁺ influx and chemotaxis in neutrophils, WT and Parp1^{-/-} bone marrow neutrophils were pretreated with 8Br-ADPR and then Ca²⁺ and chemotactic responses of these cells to fMLP was measured. As shown in FIG. 8A, the Ca²⁺ response of the fMLP-stimulated Parp1^{-/-} neutrophils was equivalent to that seen for WT neutrophils. Likewise, the chemotactic response of the Parp1^{-/-} cells to fMLP was indistinguishable from that of the WT neutrophils (FIG. 8B). However, pretreatment of either WT or Parp1^{-/-} neutrophils for 15 minutes with 8Br-ADPR blocked Ca²⁺ influx in response to fMLP and also significantly inhibited the chemotactic response of these cells to fMLP (FIG. 8A-B). In complete contrast, Cd38^{-/-} neutrophils made a very poor chemotactic response to fMLP, and preincubation with 8Br-ADPR did not further inhibit the response (FIG. 8C).

Although PARP-1 is not required to activate ADPR-gated Ca²⁺ influx in chemokine-stimulated neutrophils, it has been proposed that ADPR-gated Ca²⁺ influx in TRPM2-expressing cells exposed to H₂O₂ is controlled by PARP-1 (Fonfria, E. et al., 2004 Br J Pharmacol 143:186-192; Perraud, A. L., et al., 2005 J Biol Chem 280:6138-6148). To directly test this hypothesis in primary neutrophils, mouse bone marrow neutrophils isolated from WT, Parp1^{-/-} and Cd38−/− mice were exposed to H₂O₂ and Ca²⁺ influx in these cells was measured. A robust Ca²⁺ response in WT neutrophils treated with H₂O₂ was observed (FIG. 8D), and this response was due to influx of extracellular Ca²⁺ as it was not observed when the extracellular Ca²⁺ was chelated with EGTA. Similar results were observed when Cd38^{-/-} cells were treated with H₂O₂ (FIG. 8D). In contrast, the Ca²⁺ response in H₂O₂-treated Parp1^{-/-} neutrophils was significantly decreased (FIG. 8D). Finally, to test whether ADPR-gated Ca²⁺ influx is required for oxidant-induced Ca²⁺ entry in primary mouse neutrophils, WT and Cd38^{-/-} neutrophils with treated with 8Br-ADPR for 15 minutes and then exposed to H₂O₂. Surprizingly, 8Br-ADPR treatment did not block Ca²⁺ influx in either WT or Cd38^{-/-} neutrophils (FIG. 8D). Taken together, data indicate that oxidant-induced Ca²⁺ entry in primary mouse neutrophils is dependent on PARP-1 but can proceed in the absence of CD38 and even when the ADPR inhibitor, 8Br-ADPR, is present. In contrast, chemokine-induced Ca²⁺ entry in primary mouse neutrophils is dependent on CD38 and ADPR-gated Ca²⁺ influx but does not require PARP-1. Thus, oxidant-induced and chemokine-induced Ca²⁺ entry are mediated by distinct, yet potentially related, mechanisms.

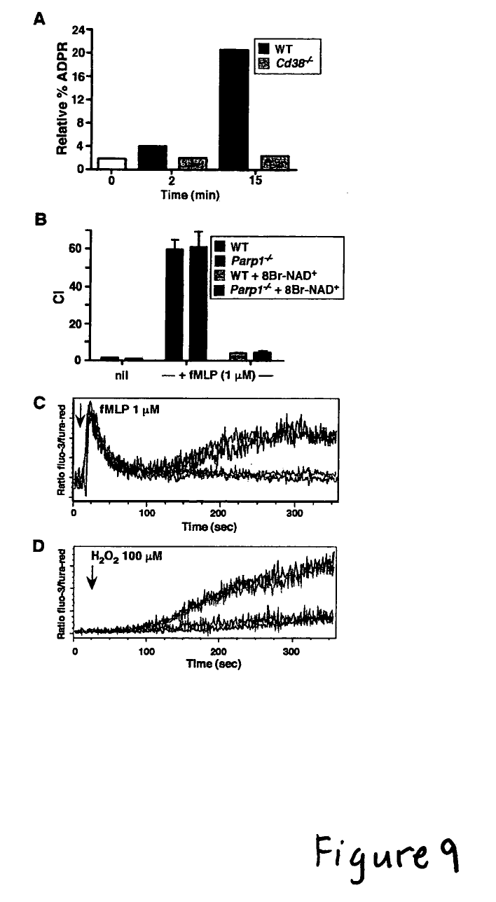

A CD38 substrate analog blocks ADPR-gated Ca²⁺ entry and chemotaxis but does not affect oxidant-induced Ca²⁺ entry. Together, the data indicated cADPR and ADPR are each needed to activate Ca²⁺ influx in chemoattractant-stimulated neutrophils and DCs and that CD38, and not PARP-1, is required for chemokine receptor signaling. It had previously been shown that treatment of either human or mouse neutrophils and DCs with a NAD⁺ analog, 8Br-NAD⁺, blocked the chemotactic responses of these cells to several chemokines and it was proposed that this inhibition was due to catabolism of the 8Br-NAD⁺ by CD38-expressing cells into 8Br-cADPR (Lund, F. E. et al., 2002 Kluwer Academic Publishers 217-240; Partida-Sanchez, S. et al., 2003 Microbes Infect 5:49-58). Since the predominant product produced by CD38 under steady state conditions is ADPR (˜98-99% of the reaction products (Schuber, F. et al., 2004 Curr Mol Med 4:249-261; Howard, M. et al, 1993 Science 262:1056-1059), it seemed more likely that the 8Br-ADPR produced by the CD38-expressing cells is responsible for blocking Ca²⁺ influx and chemotaxis. However, PARP-1 also utilizes NAD⁺ as a substrate and if 8Br-NAD⁺ was able to gain access to the interior of the cells through Connexin 43 hemi-channels as has been reported for NAD⁺ (Bruzzone, S. et al., 2001 Faseb J 15:10-12), then 8Br-ADPR could potentially be generated by the PARP-1/PARG metabolic pathway. To test this possibility, 8Br-NAD⁺ was first applied extracellularly to WT and Cd38^{-/-} neutrophils followed by measurement of the accumulation of 8Br-ADPR in the culture supernatants. Within 15 minutes of incubating CD38-expressing neutrophils with 8Br-NAD⁺, 8Br-ADPR was easily detected in the culture media (FIG. 9A). Importantly, no production of 8Br-ADPR was observed in the Cd38^{-/-} cell cultures (FIG. 9A), indicating that CD38 is the sole producer of extracellular 8Br-ADPR. To test whether 8Br-NAD⁺ could be internalized and catabolized by PARP-1 into the inhibitor 8Br-ADPR, WT and Parp1^{-/-} neutrophils were incubated with 8Br-NAD⁺ and Ca²⁺ and chemotactic responses to fMLP were measured. As shown in FIG. 9B, the chemotactic response of both WT and Parp1^{-/-} neutrophils to fMLP was inhibited in the presence of 8Br-NAD⁺. Likewise, Ca²⁺ entry in response to fMLP stimulation was significantly reduced in the 8Br-NAD⁺-treated WT and Parp1^{-/-} cells (FIG. 9C). In contrast, 8Br-NAD⁺ treatment had no effect on oxidant-induced Ca²⁺ influx in WT neutrophils (FIG. 6D). Taken together, the data show that CD38 substrate analogs can be used to selectively target ADPR/TRPM2-dependent leukocyte trafficking without affecting oxidant-induced PARP-1 dependent responses and suggest that drugs targeting the CD38/TRPM2 signaling pathway could be used to treat inflammation.

The present invention is not to be limited in scope by the specific embodiments described herein which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the claims. Various publications are cited herein, the contents of which are hereby incorporated, by reference, in their entireties.

We claim:

1. A method for inhibiting the migratory activity of TRPM2 expressing cells comprising contacting said cells with a TRPM2 inhibitor.

2. The method of claim 1 wherein said TRPM2 inhibitor is an ADPR antagonist.

3. The method of claim 2 wherein said TRPM2 inhibitor is 8Br-ADPR.

4. The method of claim 1 wherein said TRPM2 expressing cells are hematopoietic cells.

5. The method of claim 1 wherein said cells are selected from the group consisting of neutrophils, lymphocytes eosinophils, macrophages, monocytes and dendritic cells.

6. A method for inhibiting the migratory activity of TRPM2 expressing cells comprising contacting said cells with a compound that modulates TRPM2 expression.

7. The method of claim 6 wherein the compound is a short interfering nucleic acid that directs cleavage of a TRPM2RNA via RNA interference.

8. A method for identifying a compound that activates the TRPM2 cation channel comprising (i) contacting a cell expressing TRPM2 with a test compound and measuring the level of TRPM2 activity; (ii) in a separate experiment, contacting a cell expressing TRPM2 protein with a placebo or vehicle control and measuring the level of TRPM2 activity where the conditions are essentially the same as in part (i), and then (iii) comparing the level of TRPM2 activity measured in part (i) with the level of TRPM2 activity in part (ii), wherein an increased level of TRPM2 activity in the presence of the test compound indicates that the test compound is a TRPM2 activator.

9. A method for identifying a compound that inhibits the TRPM2 cation channel comprising (i) contacting a cell expressing TRPM2 with a test compound and a known activator of the TRPM2 cation channel (i.e. ADPR or a chemoattractant) and measuring the level of TRPM2 activity; (ii) in a separate experiment, contacting a cell expressing TRPM2 with a placebo or vehicle control and an activator of the TRPM2 cation channel (i.e. ADPR or a chemoattractant), where the conditions are essentially the same as in part (i) and then (iii) comparing the level of TRPM2 activity measured in part (i) with the level of TRPM2 activity in part (ii), wherein a decrease level of TRPM2 activity in the presence of the test compound indicates that the test compound is a TRPM2 inhibitor.

10. The method of claim 8 or 9 wherein said TRPM2 expressing cells also express CD38.

11. The method of claim 10 wherein step (i) and (ii) are done in the presence of a chemoattractant.

12. The method of claim 11 wherein the chemoattractant is selected from the group consisting of fMet-leu-Phe (fMLP), eotaxin, GRO-1, IP-10, SDF-1, BLC, Rantes, MIP-1□, MCP-3, MIP3□, IL-8, SLC, ELC, Lymphotactin, PAF, Ltb4, complement c5a, MCP-1, amyloid 13 peptide, serum amyloid A and histamine.

13. The method of claim 8 or 9 wherein the activity of TRPM2 is measured by assaying for changes in intracellular Ca²⁺ levels.

14. The method of claim 13 wherein Ca²⁺ levels are measured using calcium indicator dyes.

15. The method of claim 8 or 9 wherein the activity of TRPM2 is measured by assaying for changes in membrane potential.

16. The method of claim 15 wherein changes in membrane potential are measured using a voltage clamp or patch recording method.

17. The method of claim 8 or 9 wherein the activity of TRPM2 is measured by assaying for changes in cell migration.

18. A method for identifying of compound capable of inhibiting cell migration comprising identification of a TRPM2 agonist which causes desensitization of the chemoattractant receptor by depletion of intracellular calcium stores.

19. A method of treating a disorder associated with cell migration comprising administration of a compound that modulates TRPM2 channel activity.

20. The method of claim 19 wherein the cell is a hematopoietic cell.

21. The method of claim 20 wherein the disorder is selected from the group consisting of inflammation, ischemia, atherosclerosis, asthma, auto-immune disease, diabetes, allergies, infections, arthritis and organ transplant rejections.


SEQUENCE LISTING (2 sequences)

SEQ ID NO: 1 - DNA, 22 bp, Artificial Sequence
  Description of Artificial Sequence Synthetic primer
tgcctttggtgacatcgttttc

SEQ ID NO: 2 - DNA, 25 bp, Artificial Sequence
  Description of Artificial Sequence Synthetic primer
gatggccacacctcccctttccttc